(19) United States
(12) Patent Application Publication
(10) Pub. No.: US 2021/0093345 A1
(43) Pub. Date: Apr. 1, 2021

(54) TREATMENT TOOL

(71) Applicant: OLYMPUS CORPORATION, Tokyo (JP)

(72) Inventor: Seigo KASE, Sagamihara-shi (JP)

(73) Assignee: OLYMPUS CORPORATION, Tokyo (JP)

(21) Appl. No.: 17/120,978

(22) Filed: Dec. 14, 2020

Related U.S. Application Data
(63) Continuation of application No. PCT/JP2018/022970, filed on Jun. 15, 2018.

(51) Int. Cl. A61B 17/28; A61B 17/32; A61B 18/14

(52) U.S. Cl. CPC A61B 17/282; A61B 2018/00589; A61B 18/1442; A61B 17/320092

(57) ABSTRACT

A treatment tool includes: a first jaw; a second jaw that is openable and closable relative to the first jaw; a grasping member that is supported by the second jaw so as to be rotatable about a first rotation axis from a first state to a second state, and grasps living tissue in a space formed with the first jaw; and a regulating member that regulates rotation of the grasping member with respect to the second jaw to set the grasping member to one of the first state and the second state in a state in which the second jaw is opened with respect to the first jaw. The regulating member is a magnetic member that regulates rotation of the grasping member with respect to the second jaw by a magnetic force, and regulation of rotation of the grasping member is released when the living tissue is grasped.

This application is a continuation of International Application No. PCT/JP2018/022970, filed on Jun. 15, 2018, the entire contents of which are incorporated herein by reference.

### BACKGROUND

The present disclosure relates to a treatment tool.

In the related art, a treatment tool for treating living tissue by applying treatment energy to the living tissue has been known (for example, see International Publication No. WO 2017/022287).

In the treatment tool described in International Publication No. WO 2017/022287, ultrasound energy and high-frequency energy are adopted as the treatment energy. Specifically, the treatment tool grasps living tissue between a blade and a jaw that are arranged at a distal end of an ultrasound probe. Further, the treatment tool transmits ultrasound vibration from the blade to the living tissue and applies high-frequency current between the blade and the jaw, to thereby treat the living tissue.

Furthermore, in the treatment tool described in International Publication No. WO 2017/022287, the jaw is configured as described below in order to approximately evenly apply a force to the living tissue that is grasped between the blade and the jaw.

The jaw includes an arm that allows the jaw to be opened and closed with respect to the blade, and a grasping member that is supported by the arm so as to be able to oscillate and that grasps living tissue in a space formed with the blade. In other words, by allowing the grasping member to oscillate, a position at which the strongest force is to be applied to the living tissue when the living tissue is grasped between the blade and the jaw is located in an approximately center of the jaw in the longitudinal direction, rather than a proximal end side of the jaw. With this configuration, a force is applied approximately evenly to the living tissue that is grasped between the blade and the jaw.

### SUMMARY

According to one aspect of the present disclosure, there is provided a treatment tool including: a first jaw; a second jaw that is openable and closable relative to the first jaw; a grasping member that is supported by the second jaw so as to be rotatable about a first rotation axis from a first state to a second state, and grasps living tissue in a space formed with the first jaw; and a regulating member that regulates rotation of the grasping member with respect to the second jaw to set the grasping member to one of the first state and the second state in a state in which the second jaw is opened with respect to the first jaw, wherein the first state is a state in which a first region of the grasping member comes into contact with the second jaw and a second region of the grasping member is separated from the second jaw, the first region being located between the first rotation axis and a distal end of the grasping member, the second region being located between the first rotation axis and a proximal end of the grasping member, the second state is a state in which the second region comes into contact with the second jaw and the first region is separated from the second jaw, the regulating member is a magnetic member that regulates rotation of the grasping member with respect to the second jaw by a magnetic force, and regulation of rotation of the grasping member by the regulating member is released when the living tissue is grasped between the first jaw and the grasping member.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a treatment tool according to an exemplary embodiment;

FIG. 2 is a diagram illustrating a distal end side of an arm;

FIG. 3 is a diagram illustrating the distal end side of the arm;

FIG. 4 is a diagram illustrating a mounting structure of a regulating member with respect to an arm main body;

FIG. 5 is a diagram for explaining functions of the regulating member;

FIG. 6 is a diagram for explaining functions of the regulating member;

FIG. 7 is a diagram for explaining functions of the regulating member;

FIG. 8 is a diagram illustrating a regulating member according to an exemplary embodiment;

FIG. 9 is a diagram illustrating the regulating member according to an exemplary embodiment;

FIG. 10 is a diagram illustrating the regulating member according to an exemplary embodiment;

FIG. 11 is a diagram illustrating a mounting structure of a sliding member with respect to an arm main body;

FIG. 12 is a diagram illustrating a regulating member according to an exemplary embodiment;

FIG. 13 is a diagram illustrating a regulating member according to an exemplary embodiment; and

FIG. 14 is a diagram illustrating a regulating member according to an exemplary embodiment.

### DETAILED DESCRIPTION

Modes for carrying out the present disclosure (hereinafter, referred to as embodiments) will be described below with reference to the drawings. The present disclosure is not limited by the embodiments described below. In addition, in descriptions of the drawings, the same components are denoted by the same reference symbols.

### First Embodiment

Configuration of Treatment Tool

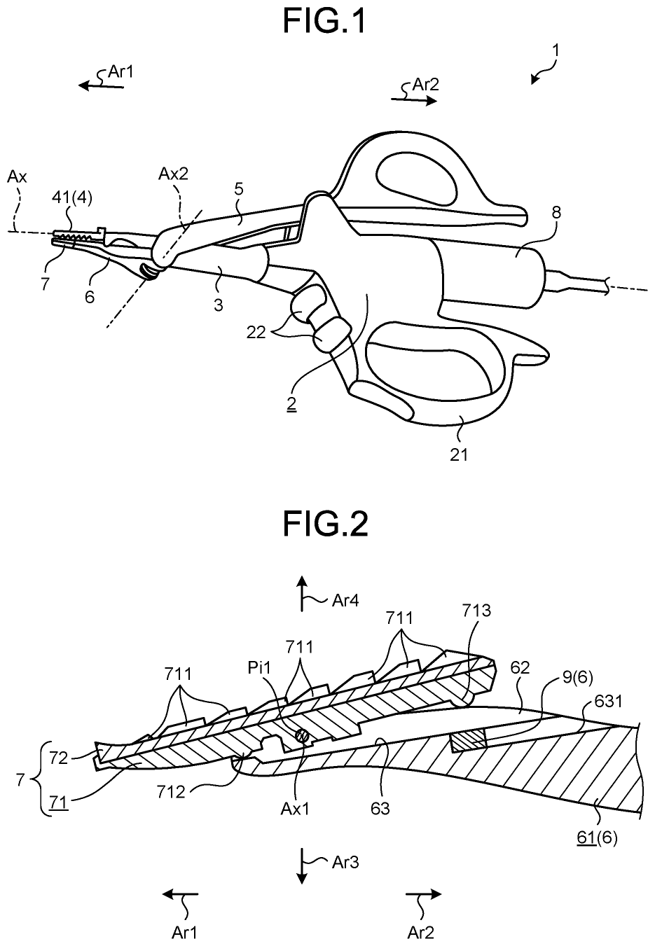

FIG. 1 is a diagram illustrating a treatment tool 1 according to an exemplary embodiment.

The treatment tool 1 applies ultrasound energy and high-frequency energy to a region to be treated (hereinafter, described as a target region) in living tissue, thereby treating the target region. Here, the treatment indicates, for example, coagulation and incision of the target region. In the first exemplary embodiment, the treatment tool 1 is a forceps-type treatment tool. As illustrated in FIG. 1, the treatment tool 1 includes a housing 2, a sheath 3, an ultrasound probe 4, a movable handle 5, an arm 6, a grasping member 7, and an ultrasound transducer 8.

As illustrated in FIG. 1, the housing 2 includes a fixed handle 21 and switches 22.

The fixed handle 21 is a portion that is to be held by an operator.

The switches 22 are arranged so as to be exposed to the outside of the housing 2, and receive output start operation performed by the operator. Further, the switches 22 output an operation signal corresponding to the output start operation to a control device (not illustrated) that is electrically connected to the treatment tool 1.

The sheath 3 corresponds to a support member according to the present disclosure. The entire sheath 3 has an approximately cylindrical shape. In the following, one side of the sheath 3 along a central axis Ax will be described as a distal end side Ar1 (FIG. 1), and the other side will be described as a proximal end side Ar2 (FIG. 1). Further, an end portion of the sheath 3 on the proximal end side Ar2 is mounted on an end portion of the housing 2 on the distal end side Ar1.

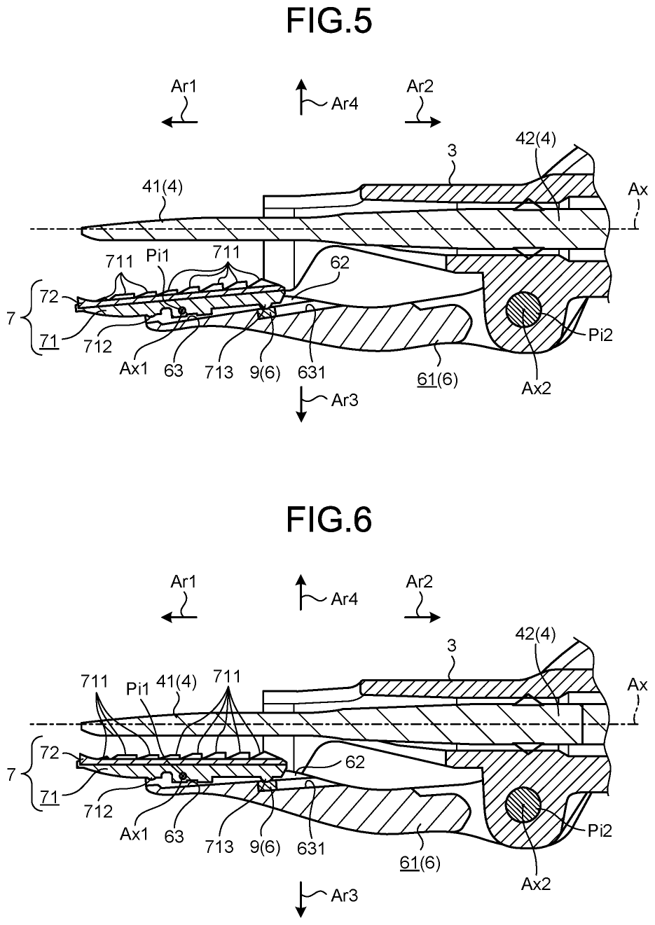

The ultrasound probe 4 corresponds to a first jaw according to the present disclosure. The ultrasound probe 4 has an elongated shape and made with a conductive material. Further, as illustrated in FIG. 1, the ultrasound probe 4 is inserted in the sheath 3 such that a blade 41 is exposed to the outside. Furthermore, the ultrasound probe 4 is supported by the sheath 3. The ultrasound probe 4 includes the blade 41 and a shaft 42 (see FIG. 5 to FIG. 7).

The blade 41 is arranged on a distal end of the shaft 42.

The shaft 42 has an elongated shape extending along the central axis Ax, and an end portion on the proximal end side Ar2 is connected to a bolted Langevin transducer (BLT) that constitutes the ultrasound transducer 8. Further, the shaft 42 transmits ultrasound vibration generated by the BLT from the end portion on the proximal end side Ar2 to the blade 41. In the first exemplary embodiment, the ultrasound vibration is longitudinal vibration that vibrates in a direction along the central axis Ax. In this case, the blade 41 vibrates at predetermined amplitude due to the longitudinal vibration of the ultrasound probe 4.

An end portion of the movable handle 5 on the distal end side Ar1 is mounted on the sheath 3 with a cylindrical second pin Pi2 (see FIG. 5 to FIG. 7). A central axis of the second pin Pi2 corresponds to a second rotation axis Ax2 (FIG. 1) according to the present disclosure. Here, the second rotation axis Ax2 is located below the central axis Ax and perpendicular to a plane including the central axis Ax in FIG. 1. Further, the movable handle 5 is extended upward from the end portion on the distal end side Ar1 in FIG. 1, and extended toward the proximal end side Ar2 so as to be located above the sheath 3 and the housing 2 in FIG. 1. Furthermore, the movable handle 5 rotates upward or downward about the second rotation axis Ax2 in FIG. 1 by operation performed by the operator.

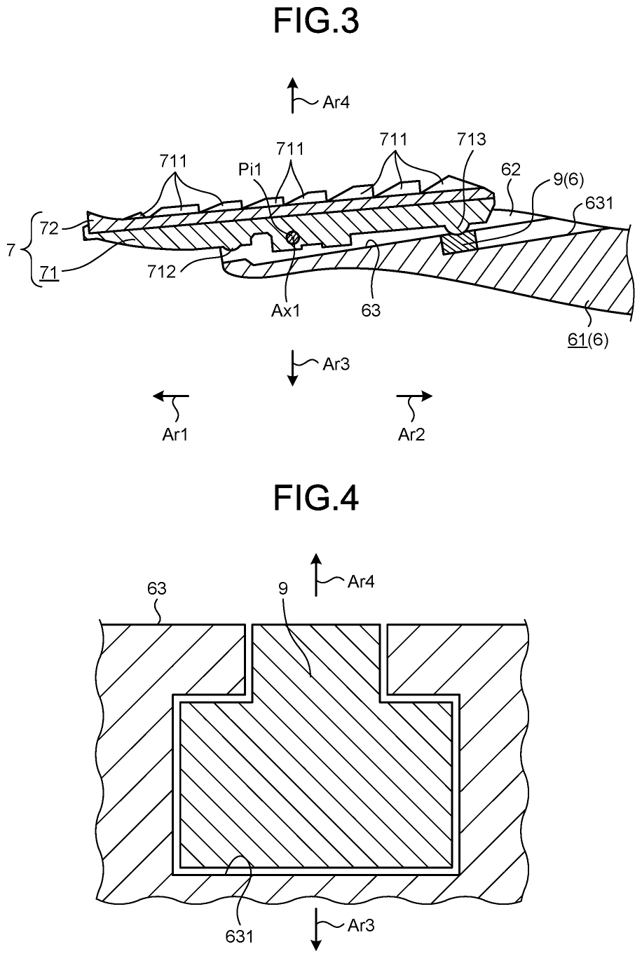

FIG. 2 and FIG. 3 are diagrams illustrating a distal end side of the arm 6. Specifically, FIG. 2 and FIG. 3 are cross-sectional views of the distal end side of the arm 6 taken along a plane that goes along the central axis Ax.

In the following, in description of configurations of the arm 6 and the grasping member 7, a lower side that is a side separated from the blade 41 in FIG. 1 will be described as a back side Ar3 (FIG. 2 and FIG. 3), and an upper side that is a side approaching the blade 41 in FIG. 1 will be described as a blade side Ar4 (FIG. 2 and FIG. 3).

The arm 6 corresponds to a second jaw according to the present disclosure. As illustrated in FIG. 2 or FIG. 3, the arm 6 includes an arm main body 61 and a regulating member 9.

The arm main body 61 is made with a conductive material, and integrally formed with the end portion of the movable handle 5 on the distal end side Ar1. Further, the arm main body 61 is extended from the end portion of the movable handle 5 on the distal end side Ar1 toward the distal end side Ar1 so as to be located on the lower side of the sheath 3 in FIG. 1. Furthermore, if the movable handle 5 rotates upward about the second rotation axis Ax2 in FIG. 1, the arm main body 61 rotates toward the back side Ar3 (lower side in FIG. 1) about the second rotation axis Ax2. Moreover, if the movable handle 5 rotates downward about the second rotation axis Ax2 in FIG. 1, the arm main body 61 rotates toward the blade side Ar4 (upper side in FIG. 1) about the second rotation axis Ax2. In other words, the arm 6 is openable and closable with respect to the blade 41.

In the arm main body 61, a pair of side wall portions 62 that protrude toward the blade side Ar4 and extend along the central axis Ax are formed on the end portion on the distal end side Ar1 as illustrated in FIG. 2 or FIG. 3. The side wall portions 62 as a pair face each other in a width direction (direction perpendicular to sheets of paper in FIG. 2 and FIG. 3) of the arm main body 61. Meanwhile, in FIG. 2 and FIG. 3, only one of the side wall portions 62 as a pair is illustrated. Further, the side wall portions 62 as a pair form a mounting concave portion 63 for mounting the grasping member 7 on an end portion of the arm main body 61 on the distal end side Ar1.

Furthermore, a cylindrical first pin Pi1 (FIG. 2 and FIG. 3) is mounted across the side wall portions 62 as a pair on the end portion of the arm main body 61 on the distal end side Ar1. A central axis of the first pin Pi1 corresponds to a first rotation axis Ax1 (FIG. 2 and FIG. 3) according to the present disclosure. Here, the first rotation axis Ax1 is located on the back side Ar3 with respect to the central axis Ax, and parallel to the second rotation axis Ax2.

FIG. 4 is a diagram illustrating a mounting structure of the regulating member 9 with respect to the arm main body 61. Specifically, FIG. 4 is a cross-sectional view of the arm main body 61 and the regulating member 9 taken along a plane perpendicular to the central axis Ax.

As illustrated in FIG. 2 to FIG. 4, a mounting groove 631 that extends from a proximal end to the distal end side Ar1 is formed on a bottom surface of the mounting concave portion 63.

The mounting groove 631 is a portion on which the regulating member 9 is mounted, and has an approximately T-shaped cross section in which a width dimension on the blade side Ar4 is smaller than a width dimension on the back side Ar3 as illustrated in FIG. 4.

The regulating member 9 has a function to regulate rotation of the grasping member 7. In the first embodiment, the regulating member 9 is a magnetic member that regulates rotation of the grasping member 7 by a magnetic force. For example, the regulating member 9 is constructed with a heat resistant neodymium magnet, a cap magnet, an electromagnet, or the like. As illustrated in FIG. 4, the regulating member 9 has an approximately T-shaped cross section that conforms to an inner surface shape of the mounting groove 631, and is mounted on the arm main body 61 by being inserted in the mounting groove 631 from a proximal end of the mounting groove 631.

Functions of the regulating member 9 will be described in detail later.

An approximately central portion of the grasping member 7 in a longitudinal direction is mounted on the arm 6 with the first pin Pi1. In other words, the grasping member 7 is supported by the arm 6 so as to be able to rotate about the first rotation axis Ax1. More specifically, the grasping member 7 is rotatable about the first rotation axis Ax1 between a first state (FIG. 2) and a second state (FIG. 3). Further, when the arm 6 is closed with respect to the blade 41, the grasping member 7 grasps a target region in a space formed with the blade 41. In other words, the grasping member 7 is configured so as to be able to oscillate about the first rotation axis Ax1, so that when the target region is grasped between the blade 41 and the grasping member 7, a position at which the strongest force is applied to the target region is located at an approximately center of the grasping member 7 in the longitudinal direction, rather than the proximal end side Ar2. With this configuration, it is possible to approximately evenly apply a force to the target region that is grasped between the blade 41 and the grasping member 7.

Here, as illustrated in FIG. 2, the first state is a state in which a first contact portion 712 that is located between the first rotation axis Ax1 and a distal end of the grasping member 7 comes into contact with the bottom surface of the mounting concave portion 63 and a second contact portion 713 that is located between the first rotation axis Ax1 and a proximal end of the grasping member 7 is separated from the regulating member 9. Further, as illustrated in FIG. 3, the second state is a state in which the second contact portion 713 comes into contact with the regulating member 9 and the first contact portion 712 is separated from the bottom surface of the mounting concave portion 63. Meanwhile, the first contact portion 712 corresponds to a first region according to the present disclosure. Further, the second contact portion 713 corresponds to a second region according to the present disclosure.

As illustrated in FIG. 2 or FIG. 3, the grasping member 7 includes a wiper jaw 71 and a resin pad 72.

The wiper jaw 71 is made with a conductive magnetic material, such as SUS430 or SUS630. The wiper jaw 71 is an elongated member, and an approximately central portion of the wiper jaw 71 in the longitudinal direction is mounted on the arm 6 with the first pin Pi1 inside the mounting concave portion 63.

As illustrated in FIG. 2 or FIG. 3, a plurality of first teeth portions 711 and a plurality of second teeth portions (not illustrated) are arranged on a surface of the wiper jaw 71 on the blade side Ar4.

The plurality of first teeth portions 711 are arranged on one end side of the wiper jaw 71 in the width direction (direction perpendicular to sheets of paper of FIG. 2 and FIG. 3) on the surface on the blade side Ar4. The first teeth portions 711 protrude toward the blade side Ar4 and are aligned along the longitudinal direction of the wiper jaw 71.

The plurality of second teeth portions are arranged on the other end side of the wiper jaw 71 in the width direction on the surface on the blade side Ar4. The plurality of second teeth portions protrude toward the blade side Ar4 and are aligned along the longitudinal direction of the wiper jaw 71, similarly to the plurality of first teeth portions 711.

Further, as illustrated in FIG. 2 or FIG. 3, the first and the second contact portions 712 and 713 are arranged on a surface of the wiper jaw 71 on the back side Ar3.

The first contact portion 712 is located on the distal end side Ar1 relative to the first rotation axis Ax1, and protrudes toward the back side Ar3.

The second contact portion 713 is located on the proximal end side Ar2 relative to the first rotation axis Ax1, and protrudes toward the back side Ar3.

The resin pad 72 is an elongated member, and is made with a resin material, such as polytetrafluoroethylene (PTFE), having electrical insulation property and biocompatibility. The resin pad 72 is fixed between the plurality of first teeth portions 711 and the plurality of second teeth portions on the surface of the wiper jaw 71 on the blade side Ar4. Further, when the arm 6 is closed with respect to the blade 41 while no living tissue is present between the blade 41 and the grasping member 7, the resin pad 72 comes into contact with the blade 41. At this time, the wiper jaw 71 does not come into contact with the blade 41.

The ultrasound transducer 8 is removably connected to the proximal end side Ar2 of the housing 2. The ultrasound transducer 8 includes a BLT that generates ultrasound vibration in accordance with supplied alternating current, although details are not illustrated in the drawings.

The treatment tool 1 as described above operates in the following manner.

An operator holds the treatment tool 1 in hand and inserts a distal end portion of the treatment tool 1 into a body cavity, such as an abdominal cavity. Then, the operator operates the movable handle 5 to open the arm 6 with respect to the blade 41, to thereby grasp a target region between the blade 41 and the grasping member 7. Thereafter, the operator presses the switches 22. Further, a control device (not illustrated) that is electrically connected to the treatment tool 1 performs control as described below in accordance with an operation signal from the switches 22.

The control device supplies high-frequency electric current between the wiper jaw 71 and the blade 41 via the arm main body 61 and the shaft 42. Specifically, the high-frequency electric current flows between the plurality of first teeth portions 711 and the blade 41 and between the plurality of second teeth portions and the blade 41. Then, the high-frequency electric current flows to the target region that is grasped between the blade 41 and the grasping member 7. In other words, high-frequency energy is applied to the target region.

Further, the control device supplies alternating-current power to the BLT included in the ultrasound transducer 8 at approximately the same time as supply of the high-frequency electric current to the space between the wiper jaw 71 and the blade 41, and causes the BLT to generate ultrasound vibration. Then, the ultrasound vibration is applied from the blade 41 to the target region that is grasped between the blade 41 and the grasping member 7. In other words, ultrasound energy is applied to the target region.

Then, Joule heat is generated in the target region due to the application of the high-frequency electric current. Further, frictional heat is generated between the blade 41 and the target region due to longitudinal vibration of the blade 41. Accordingly, the target region is incised while coagulating.

Functions of Regulating Member

Functions of the regulating member 9 will be described below.

FIG. 5 to FIG. 7 are diagrams for explaining functions of the regulating member 9. Specifically, FIG. 5 to FIG. 7 are cross-sectional views of the distal end portion of the treatment tool 1 taken along a plane that goes along the central axis Ax.

As described above, the wiper jaw 71 is made with a magnetic material. Therefore, as illustrated in FIG. 5, the regulating member 9 attracts the wiper jaw 71 due to a magnetic force in a state in which the arm 6 is opened with respect to the blade 41. Accordingly, the grasping member 7 rotates in a clockwise direction about the first rotation axis Ax1 in FIG. 5 and is set to the second state in which the second contact portion 713 comes into contact with the regulating member 9 (FIG. 5). In other words, the regulating member 9 regulates rotation of the grasping member 7 about the first rotation axis Ax1 to set the grasping member 7 to the second state in the state in which the arm 6 is opened with respect to the blade 41. Further, in the first exemplary embodiment, the regulating member 9 is arranged in a certain region, which comes into contact with the second contact portion 713, on the arm 6.

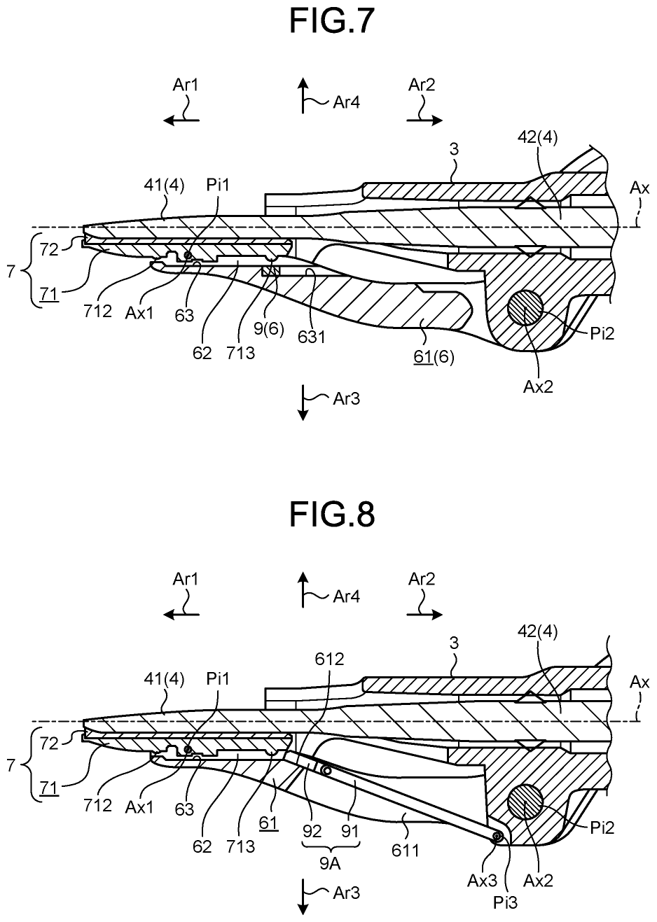

A case will be described in which the arm 6 is closed with respect to the blade 41 as illustrated in FIG. 6 and FIG. 7 from the state illustrated in FIG. 5.

In this case, in the grasping member 7 set in the second state, a certain region of the resin pad 72 on the distal end side Ar1 first comes into contact with the blade 41. Then, if the certain region of the resin pad 72 on the distal end side Ar1 is pressed by the blade 41 with a stronger force than the magnetic force of the regulating member 9, the grasping member 7 rotates in a counterclockwise direction about the first rotation axis Ax1 in FIG. 7. In other words, the second contact portion 713 is separated from the regulating member 9. Accordingly, approximately the entire surface of the resin pad 72 on the blade side Ar4 comes into contact with the blade 41 (FIG. 7). In this state, the first contact portion 712 is also separated from the bottom surface of the mounting concave portion 63. In other words, the grasping member 7 is in a neutral state in which both of the first and the second contact portions 712 and 713 are respectively separated from the bottom surface of the mounting concave portion 63 and the regulating member 9. Meanwhile, while FIG. 7 illustrates a case in which living tissue is not present between the blade 41 and the grasping member 7, even if living tissue is present, the grasping member 7 is in the neutral state. In other words, regulation of rotation of the grasping member 7 by the regulating member 9 is released when living tissue is grasped between the blade 41 and the grasping member 7.

According to the exemplary embodiment as described above, the following effects are achieved.

The treatment tool 1 according to the exemplary embodiment includes the regulating member 9 that regulates rotation of the grasping member 7 about the first rotation axis Ax1 to set the grasping member 7 to the second state in the state in which the arm 6 is opened with respect to the blade 41.

Therefore, the grasping member 7 does not oscillate in the state in which the arm 6 is opened with respect to the blade 41. In other words, when using the treatment tool 1, an operator is able to easily perform operation of turning membranous tissue by the end portion of the grasping member 7 on the distal end side Ar1 in the state in which the arm 6 is opened with respect to the blade 41. Further, it is possible to easily perform operation of grasping living tissue because the grasping member 7 does not oscillate. Therefore, according to the treatment tool 1 of the first exemplary embodiment, it is possible to improve operability.

Furthermore, according to the treatment tool 1 of the first exemplary embodiment, regulation of rotation of the grasping member 7 by the regulating member 9 is released when living tissue is grasped between the blade 41 and the grasping member 7.

Therefore, it is possible to improve operability as described above in the state in which the arm 6 is opened with respect to the blade 41, and it is possible to prevent a grasping force applied to a target region from being unbalanced due to the regulating member 9 and approximately evenly apply the force to the target region in the closing state.

Meanwhile, as the regulating member 9, it may be possible to adopt a biasing member, such as a spring, other than the magnetic member. However, if the biasing member is adopted, an excessive elastic force of the biasing member acts on the target region in the state in which the arm 6 is closed with respect to the blade 41, so that a grasping force applied to the target region may be unbalanced, which is a problem.

In contrast, in the treatment tool 1 according to the first exemplary embodiment, the regulating member 9 is the magnetic member. In other words, a magnetic force is reduced with an increase in a distance between the grasping member 7 and the regulating member 9. Therefore, in the state in which the arm 6 is closed with respect to the blade 41, it is possible to prevent the grasping force applied to the target region from being unbalanced due to the regulating member 9, and it is possible to approximately evenly apply a force to the target region.

Furthermore, in the treatment tool 1 according to the first exemplary embodiment, the regulating member 9 is arranged on the proximal end side relative to the first rotation axis Ax1, and sets the grasping member 7 to the second state in the state in which the arm 6 is opened with respect to the blade 41.

Therefore, if the arm 6 is closed with respect to the blade 41 in the state in which living tissue is not present between the blade 41 and the grasping member 7, a certain region of the resin pad 72 on the distal end side Ar1 in the grasping member 7 first comes into contact with the blade 41. In other words, it is possible to realize a structure that makes it easy to grasp living tissue by a portion of the grasping member 7 on the distal end side Ar1 when the living tissue is to be grasped.

Moreover, in the treatment tool 1 according to the first exemplary embodiment, the regulating member 9 is arranged in a certain region, which comes into contact with the second contact portion 713, on the arm 6.

Therefore, the magnetic force becomes strongest when the second contact portion 713 comes into contact with the regulating member 9. In other words, it is possible to preferably maintain regulation of rotation of the grasping member 7 by the regulating member 9.

Another exemplary embodiment will be described below. In the following description, the same components as those of the first embodiment as described above are denoted by the same reference symbols, and detailed explanation thereof will be omitted or simplified.

FIG. 8 to FIG. 10 are diagrams illustrating a regulating member 9A according to another exemplary embodiment. Specifically, FIG. 8 to FIG. 10 are diagrams corresponding to FIG. 5 to FIG. 7.

In the embodiment, as illustrated in FIG. 8 to FIG. 10, the regulating member 9A different from the regulating member 9 previously described above.

Meanwhile, in the exemplary embodiment, the arm main body 61 corresponds to the second jaw of the present disclosure because the regulating member 9 is omitted.

Further, in the exemplary embodiment, the first state is a state in which the first contact portion 712 comes into contact with the bottom surface of the mounting concave portion 63 and the second contact portion 713 is separated from the bottom surface of the mounting concave portion 63 (FIG. 10). Furthermore, in the exemplary embodiment, the second state is a state in which the second contact portion 713 comes into contact with the bottom surface of the mounting concave portion 63 and the first contact portion 712 is separated from the bottom surface of the mounting concave portion 63.

The regulating member 9A regulates rotation of the grasping member 7 by a pressing force. As illustrated in FIG. 8 to FIG. 10, the regulating member 9A includes a link 91 and a sliding member 92.

As illustrated in FIG. 8 to FIG. 10, the link 91 is an elongated member that is linearly extended, and an end portion of the link 91 on one end side is mounted on the sheath 3 with a cylindrical third pin Pi3. A central axis of the third pin Pi3 corresponds to a third rotation axis Ax3 (FIG. 8 to FIG. 10) according to the present disclosure. Here, the third rotation axis Ax3 is located below the central axis Ax in FIG. 8 to FIG. 10, located on the distal end side Ar1 relative to the second rotation axis Ax2, and parallel to the second rotation axis Ax2.

Here, the arm main body 61 according to the exemplary embodiment includes a through hole 611 that penetrates through the arm main body 61 in a vertical direction in FIG. 8 to FIG. 10 to prevent mechanical interference with the link 91 when the link 91 rotates about the third rotation axis Ax3. Further, the end portion of the link 91 on one end side is supported by the sheath 3 and the other end side is inserted in the through hole 611.

FIG. 11 is a diagram illustrating a mounting structure of the sliding member 92 with respect to the arm main body 61. Specifically, FIG. 11 is a cross-sectional view of the arm main body 61 and the sliding member 92 taken along a plane perpendicular to the central axis Ax.

Further, as illustrated in FIG. 8 to FIG. 11, a guide groove 612 that communicates with the through hole 611 and extends from the through hole 611 toward the distal end side Ar1 is formed on a surface of the arm main body 61 on the blade side Ar4.

The guide groove 612 is a portion that guides movement of the sliding member 92, and, as illustrated in FIG. 11, has an approximately T-shaped cross section in which a width dimension on the blade side Ar4 is smaller than a width dimension on the back side Ar3.

The sliding member 92 is connected to an end portion of the link 91 on the other end side. Further, as illustrated in FIG. 11, the sliding member 92 has an approximately T-shaped cross section that conforms to an inner surface shape of the guide groove 612, and is inserted in the guide groove 612 from a proximal end of the guide groove 612. Furthermore, the sliding member 92 is able to move in a direction approaching the grasping member 7 or a direction separating from the grasping member 7 while being guided by the guide groove 612.

Functions of the regulating member 9A as described above will be described below.

As illustrated in FIG. 8, in the state in which the arm 6 is closed with respect to the blade 41, the sliding member 92 is located at a position separated from the grasping member 7 inside the guide groove 612. In other words, the grasping member 7 is not pressed by the sliding member 92. Therefore, approximately the entire surface of the resin pad 72 on the blade side Ar4 comes into contact with the blade 41 (FIG. 8). In this state, the grasping member 7 is in the neutral state in which both of the first and the second contact portions 712 and 713 are separated from the bottom surface of the mounting concave portion 63. Meanwhile, while FIG. 8 illustrates a case in which living tissue is not present between the blade 41 and the grasping member 7, even if living tissue is present, the grasping member 7 is in the neutral state.

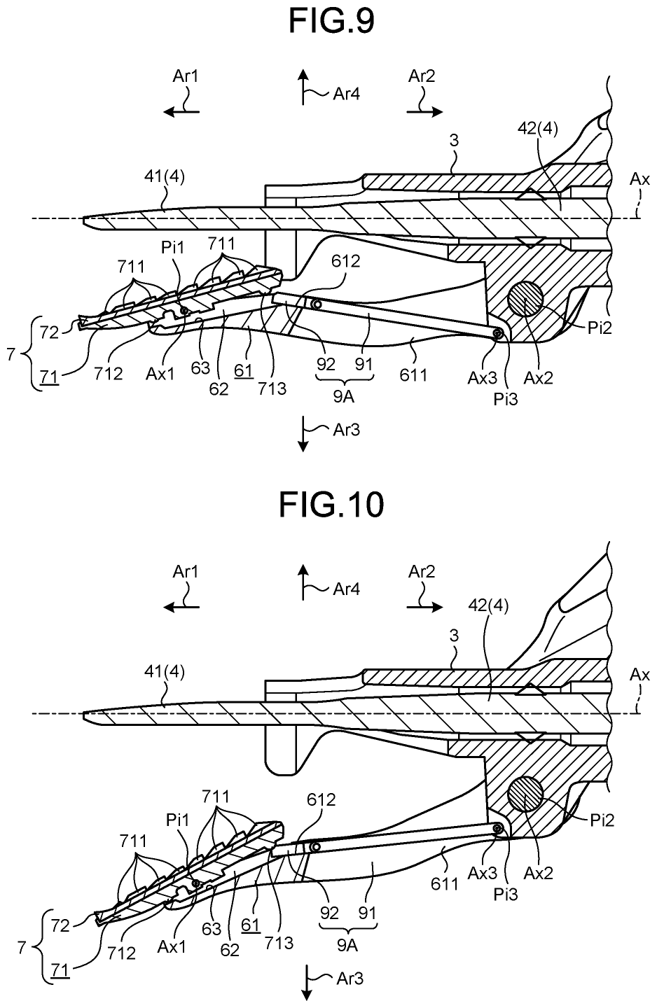

A case will be described in which the arm 6 is opened with respect to the blade 41 as illustrated in FIG. 9 and FIG. 10 from the state illustrated in FIG. 8.

Here, the second and the third rotation axes Ax2 and Ax3 are parallel to each other as described above. Therefore, if the link 91 rotates about the third rotation axis Ax3 in accordance with rotation of the arm main body 61 about the second rotation axis Ax2 in an opening direction with respect to the blade 41, the end portion of the link 91 on the distal end side Ar1 moves to the distal end side Ar1 from the state illustrated in FIG. 8. That is, the sliding member 92 moves to the distal end side Ar1 relative to the state illustrated in FIG. 8 and presses the grasping member 7 inside the guide groove 612. Accordingly, the grasping member 7 rotates in a counterclockwise direction about the first rotation axis Ax1 in FIG. 10, and is set to the first state in which the first contact portion 712 comes into contact with the bottom surface of the mounting concave portion 63 (FIG. 10). In other words, the regulating member 9A regulates rotation of the grasping member 7 about the first rotation axis Ax1 to set the grasping member 7 to the first state in the state in which the arm main body 61 is opened with respect to the blade 41.

Meanwhile, when the arm main body 61 is closed with respect to the blade 41 from the opened state, the regulating member 9A is operated reversely with respect to the above-described operation.

Even if the regulating member 9A according to the as described above is adopted, the same effects as those of regulating member 9 as described above are achieved.

Furthermore, the regulating member 9A sets the grasping member 7 to the first state in the state in which the arm main body 61 is opened with respect to the blade 41. In other words, in this state, the grasping member 7 is not able to further rotate in the counterclockwise direction in FIG. 10. Therefore, the operator is able to preferably perform operation of turning membranous tissue by the end portion of the grasping member 7 on the distal end side Ar1 in the state in which the arm main body 61 is opened with respect to the blade 41.

Meanwhile, the regulating member 9A sets the grasping member 7 to the first state in the state in which the arm main body 61 is opened with respect to the blade 41, but embodiments are not limited thereto, and it may be possible to set the grasping member 7 to the second state.

Another exemplary embodiment of the present disclosure will be described below.

In the following description, the same components as those of the first embodiment as described above are denoted by the same reference symbols, and detailed explanation thereof will be omitted or simplified.

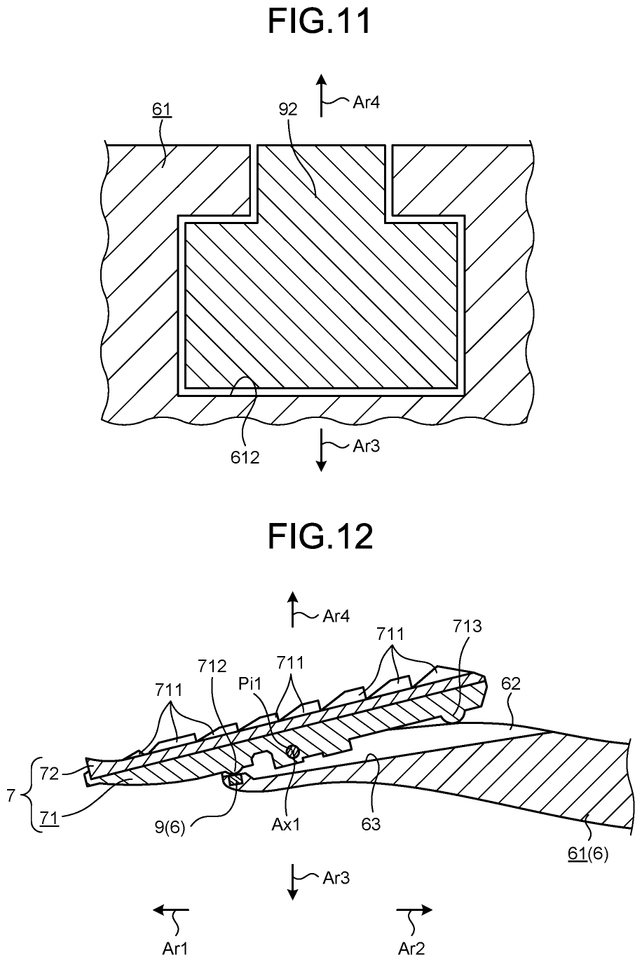

FIG. 12 is a diagram illustrating the regulating member 9. Specifically, FIG. 12 is a diagram corresponding to FIG. 2.

As illustrated in FIG. 12, a position at which the regulating member 9 is arranged is changed as compared to the first embodiment as described above.

Specifically, the regulating member 9 is arranged in a certain region, which comes into contact with the first contact portion 712, on the bottom surface of the mounting concave portion 63.

Therefore, the first state is a state in which the first contact portion 712 comes into contact with the regulating member 9, and the second contact portion 713 is separated from the bottom surface of the mounting concave portion 63 (FIG. 12). Further, the second state is a state in which the second contact portion 713 comes into contact with the bottom surface of the mounting concave portion 63, and the first contact portion 712 is separated from the regulating member 9.

Furthermore, the regulating member 9 attracts the wiper jaw 71 due to a magnetic force in the state in which the arm 6 is opened with respect to the blade 41. Accordingly, the grasping member 7 rotates in the counterclockwise direction about the first rotation axis Ax1 in FIG. 12 and is set to the first state in which the first contact portion 712 comes into contact with the regulating member 9 (FIG. 12). In other words, the regulating member 9 regulates rotation of the grasping member 7 about the first rotation axis Ax1 to set the grasping member 7 to the first state in the state in which the arm 6 is opened with respect to the blade 41.

In contrast, when the arm 6 is closed with respect to the blade 41, a certain region of the resin pad 72 on the proximal end side Ar2 first comes into contact with the blade 41. Then, if the certain region of the resin pad 72 on the proximal end side Ar2 is pressed by the blade 41 with a stronger force than the magnetic force of the regulating member 9, the grasping member 7 rotates about the first rotation axis Ax1 in the clockwise direction in FIG. 12. In other words, the first contact portion 712 is separated from the regulating member 9. Accordingly, approximately the entire surface of the resin pad 72 on the blade side Ar4 comes into contact with the blade 41. In this state, the second contact portion 713 is also separated from the bottom surface of the mounting concave portion 63. In other words, the grasping member 7 is in the neutral state in which both of the first and the second contact portions 712 and 713 are respectively separated from the regulating member 9 and the bottom surface of the mounting concave portion 63. Meanwhile, even if living tissue is present between the blade 41 and the grasping member 7, the grasping member 7 is in the neutral state. In other words, regulation of rotation of the grasping member 7 by the regulating member 9 is released when living tissue is grasped between the blade 41 and the grasping member 7.

Even if the position of the regulating member 9 is changed as described above, it is possible to achieve the same effects as those of the embodiments as described above.

Another embodiment will be described below.

In the following description, the same components as those described above will be denoted by the same reference symbols, and detailed explanation thereof will be omitted or simplified.

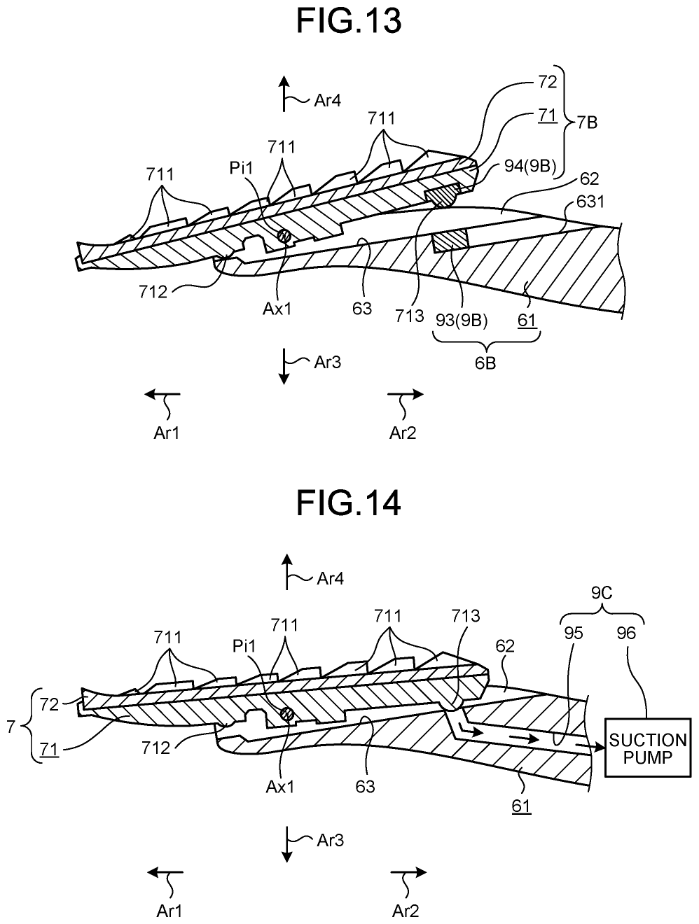

FIG. 13 is a diagram illustrating a regulating member 9B according to the fourth embodiment. Specifically, FIG. 13 is a diagram corresponding to FIG. 2.

As illustrated in FIG. 13, the regulating member 9B different from the regulating member 9 is adopted as described above.

The regulating member 9B regulates rotation of the grasping member 7 by a magnetic force, similarly to the exemplary embodiment as described above. As illustrated in FIG. 13, the regulating member 9B includes a first magnetic member 93 and a second magnetic member 94.

The first magnetic member 93 is arranged at the same position as the regulating member 9 of the exemplary embodiment as described above, and has the same shape as the regulating member 9. In other words, the first magnetic member 93 and the arm main body 61 constitute an arm 6B that corresponds to the second jaw according to the present disclosure. The first magnetic member 93 is constructed with a heat resistant neodymium magnet, a cap magnet, an electromagnet, or the like, for example.

The second magnetic member 94 is arranged on the wiper jaw 71 and constructs the second contact portion 713. In other words, the second magnetic member 94, the wiper jaw 71, and the resin pad 72 construct a grasping member 7B according to the present disclosure. The second magnetic member 94 is constructed with a heat resistant neodymium magnet, a cap magnet, an electromagnet, or the like, for example. Further, the first and the second magnetic members 93 and 94 repel to each other due to the respective magnetic forces.

Furthermore, the first and the second magnetic members 93 and 94 repel from each other in the state in which the arm 6 is opened with respect to the blade 41. Accordingly, the grasping member 7B rotates in the counterclockwise direction about the first rotation axis Ax1 in FIG. 13 and is set to the first state in which the first contact portion 712 comes into contact with the bottom surface of the mounting concave portion 63 (FIG. 13). In other words, the regulating member 9B regulates rotation of the grasping member 7B about the first rotation axis Ax1 to set the grasping member 7B to the first state in the state in which the arm 6 is opened with respect to the blade 41.

In contrast, when the arm 6B is closed with respect to the blade 41, a certain region of the resin pad 72 on the proximal end side Ar2 first comes into contact with the blade 41. Then, if the certain region of the resin pad 72 on the proximal end side Ar2 is pressed by the blade 41 with a stronger force than a magnetic repelling force of the regulating member 9B, the grasping member 7B rotates in the clockwise direction about the first rotation axis Ax1 in FIG. 13. In other words, the first contact portion 712 is separated from the bottom surface of the mounting concave portion 63. Accordingly, approximately the entire surface of the resin pad 72 on the blade side Ar4 comes into contact with the blade 41. In this state, the first and the second magnetic members 93 and 94 are separated from each other. In other words, the grasping member 7B is in the neutral state in which both of the first and the second contact portions 712 and 713 are respectively separated from the bottom surface of the mounting concave portion 63 and the first magnetic member 93. Meanwhile, even if living tissue is present between the blade 41 and the grasping member 7B, the grasping member 7B is in the neutral state. In other words, regulation of rotation of the grasping member 7B by the regulating member 9B is released when living tissue is grasped between the blade 41 and the grasping member 7B.

Even if the regulating member 9B is adopted as described above, it is possible to achieve the same effects as those of the exemplary embodiments as described above.

Another exemplary embodiment will be described below.

In the following description, the same components as those of the first embodiment as described above will be denoted by the same reference symbols, and detailed explanation thereof will be omitted or simplified.

FIG. 14 is a diagram illustrating a regulating member 9C. Specifically, FIG. 14 is a diagram corresponding to FIG. 3.

In the fifth embodiment, as illustrated in FIG. 14, the regulating member 9C different from the regulating member 9 is adopted to the first embodiment as described above.

Meanwhile, the arm main body 61 corresponds to the second jaw according to the present disclosure because the regulating member 9 is omitted.

The regulating member 9C regulates rotation of the grasping member 7 by an air suction force. As illustrated in FIG. 14, the regulating member 9C includes a suction pipeline 95 and a suction pump 96.

The suction pipeline 95 is a pipeline for circulating air, and one end of the suction pipeline 95 is arranged inside the arm main body 61 as illustrated in FIG. 14. Further, an opening of the suction pipeline 95 on the one end side is formed on the bottom surface of the mounting concave portion 63. More specifically, the opening on the one end side is arranged in a certain region, which comes into contact with the second contact portion 713, on the bottom surface of the mounting concave portion 63. Furthermore, the other end side of the suction pipeline 95 is connected to the suction pump 96.

The suction pump 96 sucks air inside the mounting concave portion 63 via the suction pipeline 95.

Further, the regulating member 9C according to the fifth embodiment attracts the wiper jaw 71 by a suction force of the suction pump 96 in the state in which the arm main body 61 is opened with respect to the blade 41. Accordingly, the grasping member 7 rotates in the clockwise direction about the first rotation axis Ax1 in FIG. 14, and is set to the second state in which the second contact portion 713 comes into contact with the bottom surface of the mounting concave portion 63 and closes the opening of the suction pipeline 95 on the one end side (FIG. 14). In other words, the regulating member 9C regulates rotation of the grasping member 7 about the first rotation axis Ax1 to set the grasping member 7 to the second state in the state in which the arm main body 61 is opened with respect to the blade 41.

In contrast, when the arm main body 61 is closed with respect to the blade 41, a certain region of the resin pad 72 on the distal end side Ar1 first comes into contact with the blade 41. Then, if the certain region of the resin pad 72 on the distal end side Ar1 is pressed by the blade 41 with a stronger force than the suction force of the suction pump 96, the grasping member 7 rotates about the first rotation axis Ax1 in the counterclockwise direction in FIG. 14. In other words, the second contact portion 713 is separated from the opening of the suction pipeline 95 on the one end side. Accordingly, approximately the entire surface of the resin pad 72 on the blade side Ar4 comes into contact with the blade 41. In this state, the first contact portion 712 is also separated from the bottom surface of the mounting concave portion 63. In other words, the grasping member 7 is in the neutral state in which both of the first and the second contact portions 712 and 713 are separated from the bottom surface of the mounting concave portion 63. Meanwhile, even if living tissue is present between the blade 41 and the grasping member 7, the grasping member 7 is in the neutral state. In other words, regulation of rotation of the grasping member 7 by the regulating member 9C is released when living tissue is grasped between the blade 41 and the grasping member 7.

Even if the regulating member 9C according to the fifth embodiment as described above is adopted, it is possible to achieve the same effects as those of the first embodiment as described above.

Meanwhile, the suction pump 96 may always be driven, or may be driven in accordance with operation performed by an operator when the arm main body 61 is opened with respect to the blade 41. Furthermore, while the suction pump 96 is adopted in the fifth embodiment as described above, embodiments are not limited thereto, and it may be possible to adopt a pump that discharges air. In other words, it may be possible to rotate the grasping member 7 about the first rotation axis Ax1 in the counterclockwise direction in FIG. 14 and set the grasping member 7 to the first state in the state in which the arm main body 61 is opened with respect to the blade 41.

### Other Embodiments

While the embodiments of the present disclosure have been described above, the present disclosure is not limited to only the first to the fifth embodiments as described above.

In the first to the fifth embodiments as described above, a configuration in which both of ultrasound energy and high-frequency energy are applied to a target region is adopted, but embodiments are not limited thereto. For example, it may be possible to adopt a configuration in which only ultrasound energy is applied to the target region, a configuration in which only high-frequency energy is applied to the target region, a configuration in which only different energy other than the ultrasound energy and the high-frequency energy is applied to the target region, or a configuration in which the configurations as described above are combined.

In the first to the fifth embodiments as described above, the treatment tool 1 is configured as a forceps-type treatment tool, but embodiments are not limited thereto, and it may be possible to adopt a configuration other than the forceps-type treatment tool (for example, the same configuration as the treatment tool described in International Publication No. WO 2017/022287).

In the first, the third, and the fourth embodiments as described above, positions at which the regulating members 9 and 9B are arranged are not limited to the positions described in the first, the third, and the fourth embodiments above, but it may be possible to adopt other positions at which a magnetic force acts.

In the exemplary embodiments as described above, a configuration in which the second jaw according to the present disclosure is opened and closed relative to the first jaw according to the present disclosure is adopted, but embodiments are not limited thereto. For example, it may be possible to adopt a configuration in which the first and the second jaws are opened and closed by moving both of the first and the second jaws. Furthermore, it may be possible to adopt a configuration in which the first jaw is opened and closed relative to the second jaw by moving the first jaw with respect to the second jaw.

According to the treatment tool of the present disclosure, it is possible to improve operability.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A treatment tool comprising:
a first jaw;
a second jaw that is configured to open and close relative to the first jaw;
a grasping member that is supported by the second jaw such that the grasping member is rotatable about a first rotation axis from a first state to a second state, and the grasping member grasps living tissue in a space formed with the first jaw; and
a regulating member that regulates rotation of the grasping member with respect to the second jaw to set the grasping member to one of the first state or the second state when the second jaw is open with respect to the first jaw, wherein:
in the first state, a first region of the grasping member contacts the second jaw and a second region of the grasping member is separated from the second jaw, the first region being located between the first rotation axis and a distal end of the grasping member, the second region being located between the first rotation axis and a proximal end of the grasping member,
in the second state, the second region comes into contact with the second jaw and the first region is separated from the second jaw,
the regulating member is a magnetic member that regulates rotation of the grasping member with respect to the second jaw by a magnetic force, and
regulation of rotation of the grasping member by the regulating member is released when the living tissue is grasped between the first jaw and the grasping member.

2. The treatment tool according to claim 1, wherein:
the regulating member is arranged between the first rotation axis and a proximal end of the treatment tool, and
the regulating member sets the grasping member to the second state when the second jaw is opened with respect to the first jaw.

3. The treatment tool according to claim 1, wherein:
the regulating member is arranged between the first rotation axis and a distal end of the treatment tool, and
the regulating member sets the grasping member to the first state when the second jaw is opened with respect to the first jaw.

4. The treatment tool according to claim 1, wherein the regulating member is arranged on at least one of the second jaw and the grasping member, in a region in which the second jaw contacts the grasping member in one of the first state and the second state.

5. The treatment tool according to claim 1, the regulating member including:
a sliding member that is configured to move towards the grasping member when the second jaw opens with respect to the first jaw, the sliding jaw being configured to move in a direction away from the grasping member when the second jaw closes with respect to the first jaw, wherein:
the sliding member is configured to regulate rotation of the grasping member with respect to the second jaw by contacting the grasping member when the second jaw is opened with respect to the first jaw.

6. The treatment tool according to claim 5, wherein the second jaw includes a guide groove that guides movement of the sliding member.

7. The treatment tool according to claim 5, further comprising:
a support member that supports the first jaw, wherein:
the second jaw is supported by the support member so that the second jaw is configured to rotate about a second rotation axis, and the second jaw opens and closes with respect to the first jaw by rotating about the second rotation axis,
the regulating member includes a link that is supported by the support member so that the regulating member is configured to rotate about a third rotation axis parallel to the second rotation axis, and that is connected to the sliding member, and
the link rotates with respect to the support member when the second jaw opens and closes with respect to the first jaw.
